(19) United States
(12) Patent Application Publication
(10) Pub. No.: US 2016/0089063 A1
(43) Pub. Date: Mar. 31, 2016

(54) COMPONENT MEASUREMENT APPARATUS AND COMPONENT MEASUREMENT METHOD

(71) Applicant: SEIKO EPSON CORPORATION, Tokyo (JP)

(72) Inventors: Kazuhiro NISHIDA, Matsumoto (JP); Tsukasa EGUCHI, Matsumoto (JP); Hitoshi TSUCHIYA, Suwa (JP)

(21) Appl. No.: 14/840,383

(22) Filed: Aug. 31, 2015

(30) Foreign Application Priority Data
Sep. 26, 2014 (JP) 2014-197034

(51) Int. Cl. A61B 5/1455; A61B 5/145

(52) U.S. Cl. CPC A61B 5/1455; A61B 5/14532

(57) ABSTRACT

The invention is provided to suppress a deterioration in the accuracy of measuring a component in a test object, which is caused by temperature variation due to irradiation with light. A blood sugar level measurement apparatus 10 includes a light emitting unit 110 that emits light toward a test object, a light receiving unit 112 that receives light that has been emitted by the light emitting unit 110 and has been reflected within or has passed through the test object, a light emission control unit 204 that performs control so as to cause the light emitting unit 110 to repeat a light emission state and a light extinction state, and a blood sugar level calculation unit 214 serving as a measurement unit that measures a component in the test object by using a result of light reception by the light receiving unit 112.

### BACKGROUND

### 1. Technical Field

The present invention relates to a component measurement apparatus that measures a component in a test object, and the like.

### 2. Related Art

As an example of an apparatus that measures a component in a test object by irradiating the test object with light, an apparatus is known that measures blood glucose concentration, or in other words, blood sugar level by utilizing a so-called light absorption phenomenon in which when a biological object is irradiated with measurement light including near infrared rays, absorption of light that has passed through a substance varies depending on the type or concentration of the substance (see, for example, JP-A-2008-35918).

Measuring a component by using a body fluid such as blood, lymph or tissue fluid as the test object has the problem of degradation of measurement accuracy due to temperature variations because the body fluid is mostly water, and water absorption characteristics strongly depend on temperature. That is, light absorbance of water is susceptible to temperature variations, and thus the light absorbance varies significantly even with a small temperature change. For this reason, there is concern that the temperature of the test object may increase by irradiation with light and the measurement accuracy may thereby deteriorate.

### SUMMARY

The invention has been made under the above-described circumstances, and an advantage of some aspects of the invention is to suppress a deterioration in the accuracy of measuring a component in a test object, which is caused by temperature variation due to irradiation with light.

A first aspect of the invention for solving the above-described problem provides a component measurement apparatus including: a light emitting unit that emits light toward a test object; a light receiving unit that receives light that has been emitted by the light emitting unit and has been reflected within or has passed through the test object; a light emission control unit that controls the light emitting unit so as to repeat a light emission state and a light extinction state; and a measurement unit that measures a component in the test object by using a result of light reception by the light receiving unit.

As another aspect of the invention, it is possible to provide a component measurement method including: controlling a light emitting unit that emits light toward a test object, so as to repeat a light emission state and a light extinction state; and measuring a component in the test object by using a result of light reception by a light receiving unit that receives light that has been emitted by the light emitting unit and has been reflected within or has passed through the test object.

According to the first aspect of the invention, the component in the test object is measured by using the result of reception of light that has been emitted and has been reflected within or has passed through the test object. At this time, control is performed so as to cause the light emitting unit emitting the emitted light to repeat the light emission state and the light extinction state. With this configuration, the temperature of the test object increases due to irradiation with the emitted light during the light emission state, but the temperature of the test object decreases due to the absence of irradiation with light during the light extinction state. As a result, it is possible to suppress an increase in the temperature of the test object due to irradiation with light, and improve the accuracy of measuring the component in the test object, as compared to the case of continuous irradiation with light.

In addition, as a second aspect of the invention, it is possible to provide, when performing control so as to repeat the light emission state and the light extinction state, the component measurement apparatus according to the first aspect of the invention, wherein the light emission control unit performs control such that a ratio of duration of the light emission state to a total duration of the light emission state and the light extinction state is within a range of 0.1 or more and less than 1.0.

A third aspect of the invention provides the component measurement apparatus according to the second aspect of the invention, further including: a temperature measurement unit that measures a temperature of the test object by using a light extinction detected value, which is a result of light reception by the light receiving unit during the light extinction state, wherein the light emission control unit controls the ratio according to the measured temperature.

According to the third aspect of the invention, the ratio of the duration of the light emission state to the total duration of the light emission state and the light extinction state is controlled according to the temperature of the test object. With this configuration, if, for example, the temperature of the test object is high, the ratio is controlled such that the duration of the light extinction state becomes relatively long so as to facilitate a decrease in the temperature of the test object. If the temperature of the test object is low, the ratio is controlled such that the duration of the light extinction state becomes relatively short so as to facilitate an increase in the temperature of the test object. Accordingly, control that maintains the temperature of the test object at a predetermined temperature is possible. As a result, it is possible to improve the accuracy of measuring the component in the test object.

Also, in this case, as a fourth aspect of the invention, it is possible to provide the component measurement apparatus according to any one of the first to third aspects of the invention, wherein the light emission control unit performs control such that duration of a single instance of the light emission state is within a range from 0.01 seconds to 10 minutes.

A fifth aspect of the invention provides the component measurement apparatus according to any one of the first to fourth aspects of the invention, further including: a correction unit that corrects a light emission detected value, which is a result of light reception by the light receiving unit during the light emission state, by using a light extinction detected value, which is a result of light reception by the light receiving unit during the light extinction state.

According to the fifth aspect of the invention, the light emission detected value is corrected by using the light extinction detected value. With this configuration, it is possible to further improve the accuracy of measuring the component in the test object.

A sixth aspect of the invention provides the component measurement apparatus according to any one of the first to fifth aspects of the invention, wherein the test object is blood in a biological object, the light emitting unit emits light including near infrared rays, and the measurement unit acquires a blood sugar level in the blood.

According to the sixth aspect of the invention, it is possible to improve the accuracy of measuring the blood sugar level in the blood.

### BRIEF DESCRIPTION OF THE DRAWINGS

The invention will be described with reference to the accompanying drawings, wherein like numbers reference like elements.

FIG. 1 shows an example of a configuration of a blood sugar level measurement apparatus.

FIGS. 2A and 2B show examples of a configuration of a sensor module.

FIG. 3 is a diagram illustrating inhibition of an increase in the temperature of a biological object by intermittent irradiation.

FIG. 4 is a diagram showing a functional configuration of the blood sugar level measurement apparatus.

FIG. 5 shows an example of a data configuration of an intermittent irradiation setting table.

FIG. 6 is a flowchart illustrating blood sugar level measurement processing.

### DESCRIPTION OF EXEMPLARY EMBODIMENTS

Outer Configuration

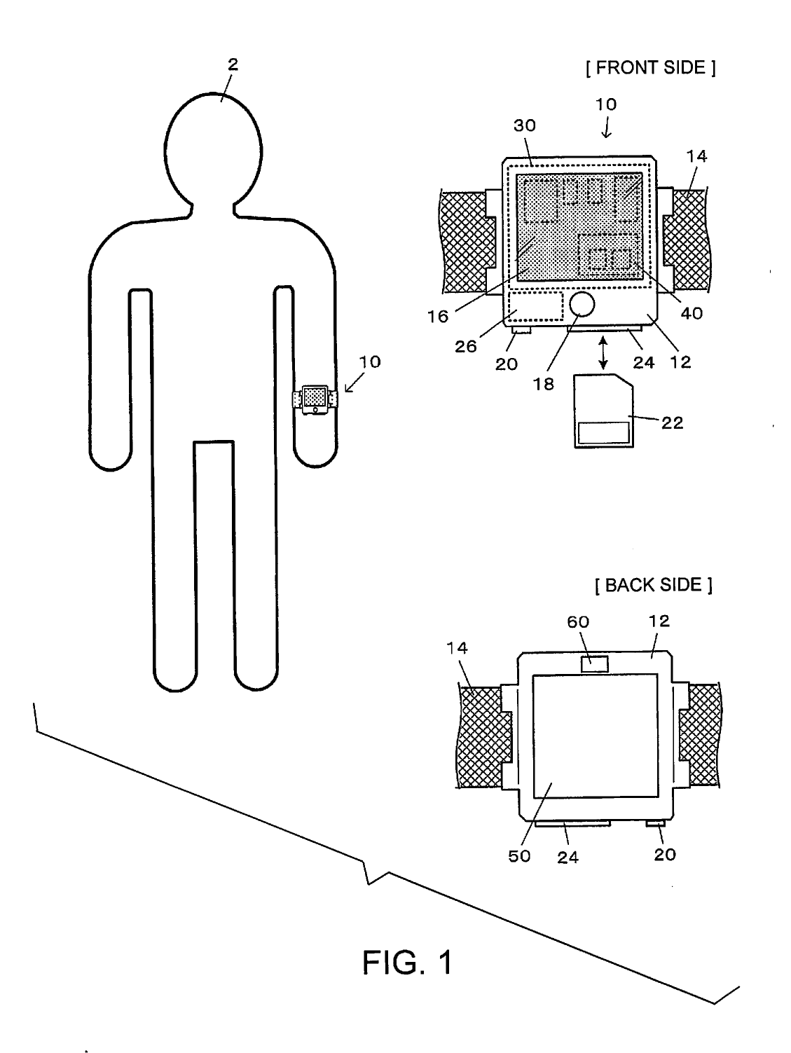

FIG.1 shows an example of a configuration of a blood sugar level measurement apparatus 10 according to an embodiment of the invention. The blood sugar level measurement apparatus 10 is an apparatus that noninvasively measures blood sugar level, which is glucose concentration, in the blood of a user 2 by using light, and is an example of a component measurement apparatus that measures blood sugar level in the blood used as a test object. The present embodiment is an example of application of the invention, and thus the invention can be applied to any other embodiment. For example, protein or lipid may be used as the component to be measured, and lymph or tissue fluid may be used as the test object, instead of blood.

As shown in FIG. 1, the blood sugar level measurement apparatus 10 is in the form of a watch, and includes a main body case 12 and a fixing band 14 for fitting and fixing the main body case 12 to a measurement area of the user 2 such as the wrist or arm. The fixing band 14 can be, for example, magic tape®.

On the front side (the side that faces the outside when worn by the user 2) of the main body case 12, a touch panel 16 and an operation switch 18 are provided. By using the touch panel 16 and the operation switch 18, the user 2 can input a measurement start instruction, and a result of measurement is displayed on the touch panel 16.

On a side surface of the main body case 12, a communication device 20 for performing communication with external apparatuses and a reader/writer 24 for a memory card 22 are provided. The communication device 20 can be implemented by a jack for connecting a wire cable, or by a wireless communication module for performing wireless communication and an antenna thereof. The memory card 22 is a data-rewritable non-volatile memory such as a flash memory, a ferroelectric random access memory (FeRAM), or a magnetoresistive random access memory (MRAM).

On the back side of the main body case 12, a sensor module 50 and a temperature sensor 60 are provided so as to be capable of coming into contact with a skin surface of the user 2. The sensor module 50 is a measurement device that emits measurement light to the skin surface of the user 2 and receives reflected/transmitted light, and can be a thin image sensor with a built-in light source. The temperature sensor 60 measures the temperature of the skin surface of the user 2. The temperature sensor 60 can be, for example, a sensor that uses a flexible substrate having a chip thermistor or a thermistor pattern printed thereon, a platinum resistance temperature detector and the like, or a sensor that uses a thermocouple element, a PN junction element, a diode and the like.

Furthermore, the main body case 12 includes therein a rechargeable battery 26 and a control substrate 30. The battery 26 may be charged with the use of a cradle via an electric contact provided on the back side of the main body case 12 placed in the cradle connected to a household power source, or may be wirelessly charged.

On the control substrate 30, a central processing unit (CPU), a main memory, a measurement data memory, a touch panel controller, a sensor module controller, and a temperature sensor controller are mounted. The main memory is a storage medium capable of storing programs and initial data, as well as storing CPU computed values, and can be implemented by a random access memory (RAM), a read only memory (ROM), a flash memory or the like. The programs and initial setting data may be stored in the memory card 22. The measurement data memory is a storage medium for storing measurement data, and can be implemented by a data-rewritable non-volatile memory such as a flash memory, a ferroelectric random access memory (FeRAM), or a magnetoresistive random access memory (MRAM). The measurement data may be stored in the memory card 22.

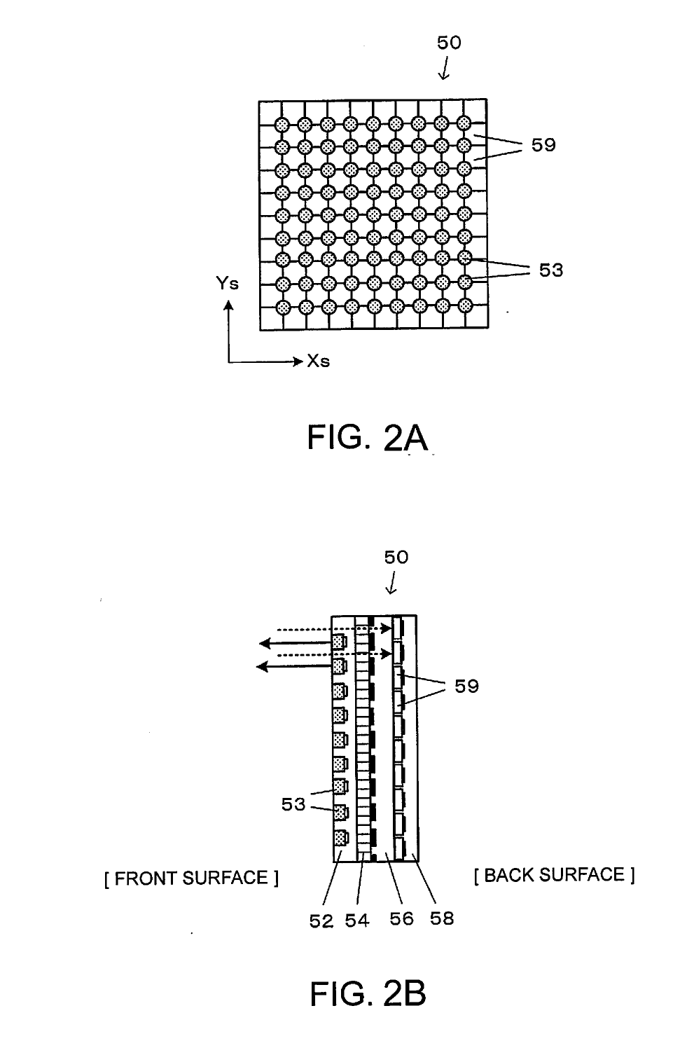

FIGS. 2A and 2B are diagrams showing a schematic configuration of the sensor module 50. FIG. 2A is a plan view, and FIG. 2B is a cross sectional view. The sensor module 50 is an optical sensor in which a light emitting layer 52 including a large number of light emitting elements 53 arranged in a two-dimensional planar array, a light blocking layer 54 that selectively blocks light other than the light travelling toward a light receiving layer 58, a spectroscopic layer 56 that selectively allows near infrared rays to pass therethrough, and the light receiving layer 58 including a large number of light receiving elements 59 arranged in a two-dimensional planar array are laminated. The sensor module 50 is provided on the back side of the main body case 12 such that its front surface (the side on which the light emitting layer 52 is provided) faces the skin surface of the user 2 when worn by the user 2.

The light emitting elements 53 constitute a light emitting unit that emits measurement light, and can be implemented by, for example, light emitting diodes (LEDs), organic light-emitting diodes (OLEDs), or the like. In the present embodiment, in order to measure blood sugar level (glucose concentration in the blood), it is desirable that the light emitting elements 53 are elements capable of emitting light including near infrared rays having skin penetration properties. The light emitting elements 53 may all have the same wavelength, or, for example, three types of light emitting elements having different wavelengths may be arranged in regularity. In the latter case, a light absorption spectrum can be obtained by driving the light emitting elements of each wavelength in a time-division manner.

The light receiving elements 59 constitute a light receiving unit that receives transmitted light or reflected light of the measurement light and outputs an electric signal according to the amount of light received, and can be implemented by, for example, an image sensor such as a charge-coupled device (CCD) or a complementary metal oxide semiconductor image sensor (CMOS). A single light receiving element includes a plurality of elements that separate light into components of wavelengths required for measurement.

The light emitting elements 53 of the light emitting layer 52 and the light receiving elements 59 of the light receiving layer 58 are arranged in a matrix defined by a common Xs-Ys orthogonal coordinate system. The light emitting elements 53 and the light receiving elements 59 are arranged such that the arrangement spacing is the same in Xs and Ys axis directions, but they are staggered on the Xs-Ys plane. In other words, the light emitting layer 52 and the light receiving layer 58 are laminated such that the positions of the light emitting elements 53 and the light receiving elements 59 in the Xs and Ys axis directions are offset from each other by a predetermined length. With this configuration, the light that has passed through the biological tissue of the user 2 and the light reflected within the biological tissue (hereinafter referred to as “reflected/transmitted light” where appropriate) can reach the light receiving elements 59.

The arrangement spacing of the light emitting elements 53 in the light emitting layer 52 and the arrangement spacing of the light receiving elements 59 in the light receiving layer 58 can be set as appropriate. For example, the arrangement spacing is preferably 1 to 500 μm. From the viewpoint of manufacturing cost and measurement accuracy, the arrangement spacing may be set to, for example, 50 to 200 μm. The configuration is not limited to the configuration in which the light emitting layer 52 and the light receiving layer 58 are laminated, and the light emitting elements 53 and the light receiving elements 59 may be arranged side by side.

Principle

(A) Measurement of Blood Sugar Level

In order to perform a blood sugar level measurement, the blood sugar level measurement apparatus 10 is fitted and fixed by the fixing band 14 such that the sensor module 50 is in close contact with the skin surface of the user 2. As a result of the sensor module 50 being brought into close contact with the skin surface, the surrounding ambient light other than the measurement light is prevented from entering the surface of the sensor module 50 that is in close contact with the skin surface, and thus a factor that cause a deterioration of the measurement accuracy can be suppressed. Then, a blood vessel within the biological tissue directly below the sensor module 50 is set as the blood vessel to be measured, a light absorption spectrum is obtained by receiving light including transmitted light of measurement light that has passed through the blood vessel, and then a blood sugar level is estimated/computed by using a calibration curve indicating a relationship between pre-set blood sugar level (glucose concentration in the blood) and light absorbance.

(B) Intermittent Irradiation

A feature of the present embodiment is to perform intermittent irradiation that periodically repeats a light emission state in which the measurement light is emitted and a light extinction state in which the measurement light is not emitted, so as to suppress an increase in the temperature of the biological object due to irradiation with the measurement light and improve the measurement accuracy. As described above, the blood sugar level measurement is performed based on the light absorption spectrum of light that has passed through the blood in the blood vessel. Water is the component that accounts for the largest proportion of the blood. It is known that the light absorption spectrum of water strongly depends on temperature. That is, if the temperature of the biological object, or in other words, the temperature of the blood in the blood vessel increases due to irradiation with the measurement light, the obtained light absorption spectrum varies, and as a result, the accuracy of blood sugar level measurement deteriorates. For this reason, in the present embodiment, intermittent irradiation that repeatedly causes the light emitting elements 53 to emit and not emit light is performed to suppress an increase in the temperature of the biological object due to irradiation with the measurement light.

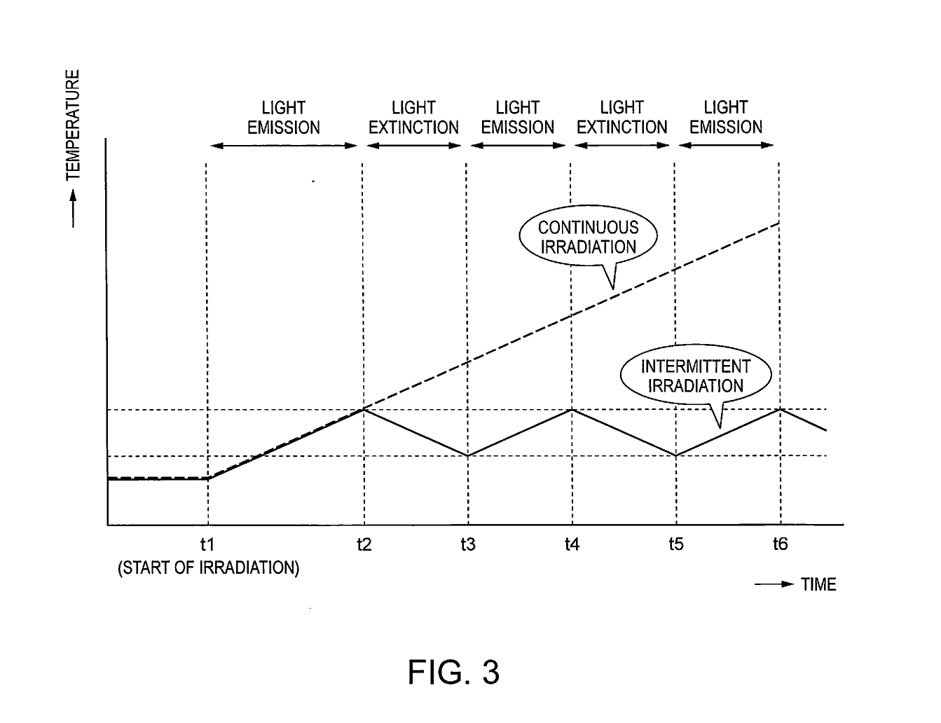

FIG. 3 is a diagram illustrating inhibition of an increase in the temperature of the biological object by intermittent driving. In FIG. 3, the horizontal axis indicates time, and the vertical axis indicates the temperature of the biological object. At time t1, light emission of the light emitting element 53 is started. The broken line indicates an example of a change in the temperature of the biological object in the case where continuous irradiation is performed, and the solid line indicates an example of a change in the temperature of the biological object in the case where intermittent irradiation is performed.

When the light emitting elements 53 are illuminated to emit measurement light, the temperature of the biological object increases. When, on the other hand, the light emitting elements 53 are not illuminated and measurement light is not emitted, the temperature of the biological object decreases. That is, as indicated by the broken line, in the case where continuous irradiation is performed, the temperature of the biological object increases in proportion to the elapsed time. Likewise, as indicated by the solid line, in the case where intermittent irradiation is performed, the temperature of the biological object repeatedly increases and decreases according to the periodical repetition between the light emission state and the light extinction state of the light emitting elements 53. The example shown in FIG. 3 illustrates that as a result of the repeating cycle between the light emission state and the light extinction state being constant, the range of variation in the temperature of the biological object is within a predetermined temperature range.

The variation in the temperature of the biological object due to intermittent irradiation is determined primarily by the durations of light emission time Ta and light extinction time Tb of the light emitting elements 53. In the present embodiment, the ratio of the light emission time Ta to the total duration of the light emission time Ta and the light extinction time Tb is defined as duty ratio D (=Ta/(Ta+Tb)). The duty ratio D takes a value that satisfies 0.0<D<1.0. A duty ratio D of 1.0 indicates that the light extinction time Tb is zero, or in other words, corresponds to a continuous irradiation state. A duty ratio D of 0.0 indicates that the light emission time Ta is zero, or in other words, corresponds to a non-irradiation state.

Then, the duty ratio D is changed according to the temperature of the skin surface of the user 2 measured by the temperature sensor 60, so as to maintain the temperature of the biological object within a predetermined temperature range. At this time, the light extinction time Tb is changed so as to change the duty ratio, with the light emission time Ta being fixed. To be specific, if the measured temperature is high, the duty ratio is reduced so as to extend the light extinction time Tb. If the measured temperature is low, the duty ratio is increased so as to shorten the light extinction time Tb. Here, the light emission time Ta is set to be long enough to sufficiently obtain a light absorption signal, and, to be specific, is set within a range from 0.01 to 600 seconds. By doing so, if the measured temperature is high, the temperature of the biological object is decreased gradually by extending the light extinction time. Conversely, if the measured temperature is low, the temperature of the biological object is increased gradually by shortening the light extinction time.

(C) Correction of Detected Value

When the blood sugar level measurement apparatus 10 is appropriately fit, the sensor module 50 is in close contact with the skin surface, and thus the light receiving elements 59 do not receive light during the light extinction state. There is, however, a possibility that the detected values of the light receiving elements 59 may include a value resulting from a small amount of light received. Here, such light is called noise. A few causes of noise that is included in the detected value can be considered. A first cause that can be considered is temperature dependence of photodiodes serving as the light receiving elements 59. Another cause that can be considered is electric noise and the like that occur on an electronic circuit. Also, it is known that cellular activity in a biological object can produce a very small amount of light, and this is also considered as noise.

In the present embodiment, in order to suppress noise as described above, a light emission detected value, which is the detected value detected by the light receiving elements 59 during the light emission state, is corrected by using a light extinction detected value, which is the detected value detected by the light receiving elements 59 during the light extinction state, and then blood sugar level is measured based on the corrected light emission detected value. To be specific, the light emission detected value is corrected by subtracting the light extinction detected value from the light emission detected value. By doing so, noise and the like caused by the temperature dependence of the light receiving elements 59 can be removed.

Functional Configuration

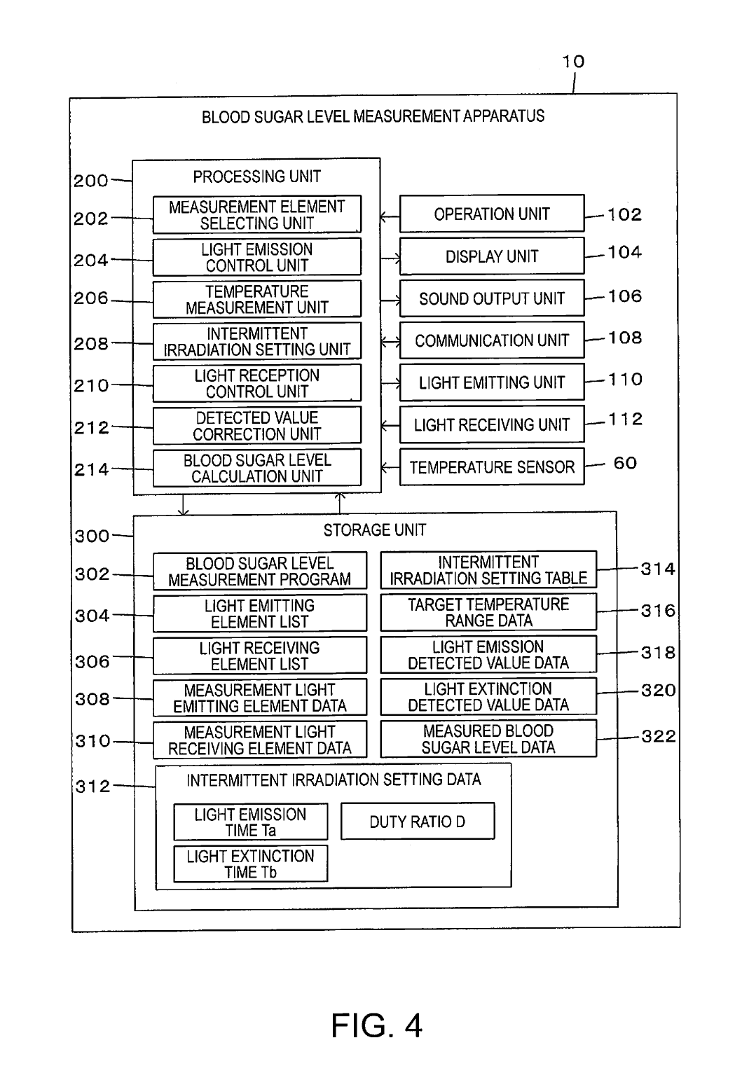

FIG. 4 is a diagram showing a functional configuration of the blood sugar level measurement apparatus 10. As shown in FIG. 4, the blood sugar level measurement apparatus 10 includes an operation unit 102, a display unit 104, a sound output unit 106, a communication unit 108, a light emitting unit 110, a light receiving unit 112, a temperature sensor 60, a processing unit 200, and a storage unit 300.

The operation unit 102 is an input device such as a button switch, a touch panel, and various types of sensors, and outputs an operational signal according to the operation made on the processing unit 200. With the use of the operation unit 102, various types of instructions such as an instruction to start a blood sugar level measurement is input. In FIG. 1, the operation switch 18 and the touch panel 16 correspond to the operation unit 102.

The display unit 104 is a display device such as a liquid crystal display (LCD), and provides various types of display screens based on a display signal from the processing unit 200. Results of measurement and the like are displayed on the display unit 104. In FIG. 1, the touch panel 16 corresponds to the display unit 104.

The sound output unit 106 is a sound output device such as a speaker, and outputs various types of sounds based on a sound signal from the processing unit 200. The sound output unit 106 outputs annunciation sounds informing the start and end of blood sugar level measurement, and the like.

The communication unit 108 is a communication device such as a wireless communication device, a modem, a wire communication cable jack or a control circuit, and implements communication with external devices by connecting to a communication line. In FIG. 1, the communication device 20 corresponds to the communication unit 108.

The light emitting unit 110 includes a large number of light emitting elements 53 that are arranged in a two-dimensional planar array. The light emitting layer 52 of the sensor module 50 shown in FIGS. 2A and 2B corresponds to the light emitting unit 110. The arrangement positions of the light emitting elements 53 (to be specific, the position coordinates of the light emitting elements 53 in the Xs-Ys coordinate system) are stored as a light emitting element list 304.

The light receiving unit 112 includes a large number of light receiving elements 59 that are arranged in a two-dimensional planar array. The light receiving layer 58 of the sensor module 50 shown in FIGS. 2A and 2B corresponds to the light receiving unit 112. The arrangement positions of the light receiving elements 59 (to be specific, the positions of the light receiving elements 59 in the Xs-Ys coordinate system) are stored as a light receiving element list 306.

The processing unit 200 can be implemented by, for example, a microprocessor such as a CPU or a graphics processing unit (GPU), or an electronic component such as an application specific integrated circuit (ASIC) or an IC memory, and executes various types of computation processing operations based on predetermined programs and data, as well as an operational signal from the operation unit 102 and the like, so as to control operations of the blood sugar level measurement apparatus 10. In FIG. 1, the control substrate 30 corresponds to the processing unit 200. Also, the processing unit 200 includes a measurement element selecting unit 202, a light emission control unit 204, a temperature measurement unit 206, an intermittent irradiation setting unit 208, a light reception control unit 210, a detected value correction unit 212, and a blood sugar level calculation unit 214.

The measurement element selecting unit 202 selects light emitting elements 53 and light receiving elements 59 for use in a blood sugar level measurement. To be specific, all of the light emitting elements 53 of the light emitting unit 110 are caused to simultaneously emit light, so as to cause all of the light receiving elements 59 of the light receiving unit 112 to receive light (to perform image capturing) and thereby to generate a luminance image resulting from the received light, or in other words, a biological object image. Next, a positional pattern of blood vessels is acquired from the generated biological object image, and then blood vessel areas to be subjected to measurement are selected. Then, with respect to each blood vessel area to be subjected to measurement, light emitting elements 53 and light receiving elements 59 to be used in measurement are selected such that at a position substantially center of the blood vessel area, it is possible to obtain a large amount of measurement light that has been emitted from the light emitting elements 53, has passed through the blood vessel area and has been received by the light receiving elements 59.

The light emitting elements 53 (measurement light emitting elements) and light receiving elements 59 (measurement light receiving elements) selected by the measurement element selecting unit 202 are respectively stored as measurement light emitting element data 308 and measurement light receiving element data 310.

The light emission control unit 204 can perform control so as to selectively cause the plurality of light emitting elements 53 of the light emitting unit 110 to emit light. Also, the light emission control unit 204 can perform control so as to cause measurement light emitting elements selected from among the plurality of light emitting elements 53 of the light emitting unit 110 to perform intermittent irradiation that periodically repeats a light emission state and a light extinction state. To be specific, the light emission control unit 204 performs control so as to cause the measurement light emitting elements to repeatedly perform light emission during the light emission time Ta set as intermittent irradiation setting data 312 and light extinction during the light extinction time Tb set as the intermittent irradiation setting data 312.

The intermittent irradiation setting data 312 is data in which parameters for intermittent irradiation are set, and includes the light emission time Ta, the light extinction time Tb, and the duty ratio D that is determined from the light emission time Ta and the light extinction time Tb.

The temperature measurement unit 206 measures the temperature of the skin surface of the user 2 measured by the temperature sensor 60 as the body temperature of the user 2.

The intermittent irradiation setting unit 208 sets parameters for intermittent irradiation performed by the light emission control unit 204. To be specific, the intermittent irradiation setting unit 208 compares the temperature measured by the temperature measurement unit 206 with a predetermined target temperature range, and in response to the result of comparison, changes the duty ratio in accordance with an intermittent irradiation setting table 314. The target temperature range refers to a target range of body temperatures of the user, and the lower limit temperature and the upper limit temperature of the target temperature range are set by, for example, an external instruction given via the operation unit 102. The target temperature range is stored as target temperature range data 316.

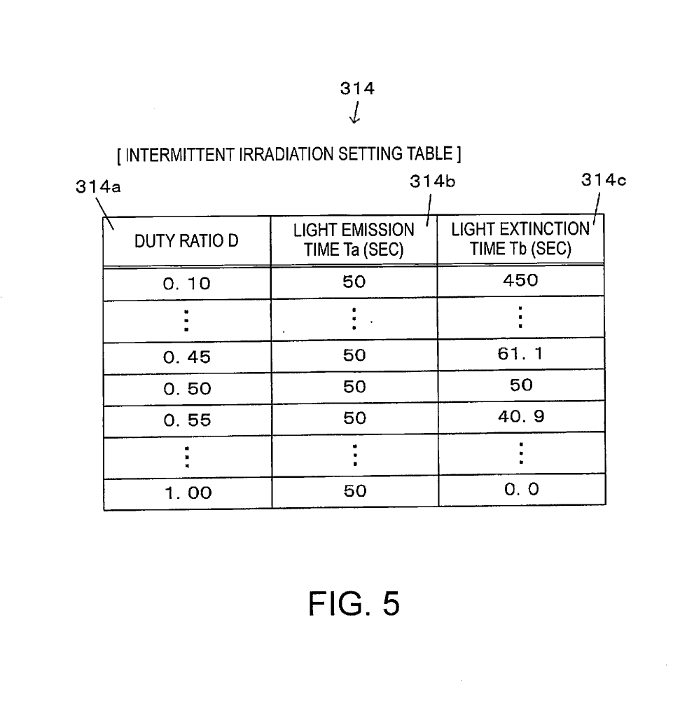

FIG. 5 is a diagram showing an example of a data configuration of the intermittent irradiation setting table 314. As shown in FIG. 5, the intermittent irradiation setting table 314 stores therein duty ratio 314a, light emission time 314b and light extinction time 314c in association with each other. In FIG. 5, the duty ratio 314a is set in increments of a predetermined change rate ΔD (=0.05) within a predetermined range (0.1 or more and less than 1.0). The light emission time 314b is fixed. Based on the duty ratio 314a and the light emission time 314b, the corresponding light extinction time 314c is determined.

To be specific, the intermittent irradiation setting unit 208 does not change the duty ratio D if the measured temperature is within the target temperature range. If the measured temperature is above the target temperature range, the intermittent irradiation setting unit 208 changes the duty ratio D so as to be smaller than the current value by a predetermined change rate ΔD. If the measured temperature is below the target temperature range, the intermittent irradiation setting unit 208 changes the duty ratio D so as to be greater by the predetermined change rate ΔD.

It is of course possible to, if the measured temperature is within the target temperature range, perform control so as to reduce the duty ratio D as the measured temperature approaches the upper limit of the range, and increase the duty ratio D as the measured temperature approaches the lower limit of the range.

The light reception control unit 210 outputs a detected value according to the amount of light received by each of the plurality of light receiving elements 59 of the light receiving unit 112. Of the detected values of the light receiving elements 59, those obtained during the light emission state are stored as light emission detected value data 318, and those obtained during the light extinction state are stored as light extinction detected value data 320.

The detected value correction unit 212 performs, with respect to each measurement light receiving element, correction of the light emission detected value by subtracting the light extinction detected value from the light emission detected value.

The blood sugar level calculation unit 214 calculates a glucose concentration in the blood, or in other words, a blood sugar level based on the light emission detected value of the measurement light receiving element corrected by the detected value correction unit 212. To be specific, the blood sugar level calculation unit 214 calculates a transmission rate per wavelength X based on the light emission detected value so as to generate a light absorption spectrum. At this time, if there are a plurality of measurement light receiving elements, a light absorption spectrum is generated with respect to each of the plurality of measurement light receiving elements, and the generated light absorption spectrums are averaged to obtain an averaged light absorption spectrum. Then, a blood sugar level is calculated (estimated) from the light absorption spectrum by using a calibration curve that indicates a relationship between pre-set glucose concentration in the blood and light absorbance. For example, the blood sugar level is calculated from the light absorption spectrum by using an analysis method such as multiple regression analysis, principal component regression analysis, PLS regression analysis or independent component regression analysis. The blood sugar level calculated by the blood sugar level calculation unit 214 is stored as measured blood sugar level data 322.

The storage unit 300 is a storage device such as a ROM, a RAM or a hard disk, and stores therein a program, data and the like for the processing unit 200 to collectively control the blood sugar level measurement apparatus 10. The storage unit 300 is used as a work area for the processing unit 200, and thus the results of computation performed by the processing unit 200, operation data from the operation unit 102, and the like are temporarily stored. In FIG. 1, the main memory and the measurement data memory mounted on the control substrate 30 correspond to the storage unit 300. In the storage unit 300, a blood sugar level measurement program 302, the light emitting element list 304, the light receiving element list 306, the measurement light emitting element data 308, the measurement light receiving element data 310, the intermittent irradiation setting data 312, the intermittent irradiation setting table 314, the target temperature range data 316, the light emission detected value data 318, the light extinction detected value data 320, and the measured blood sugar level data 322 are stored.

Processing Flow

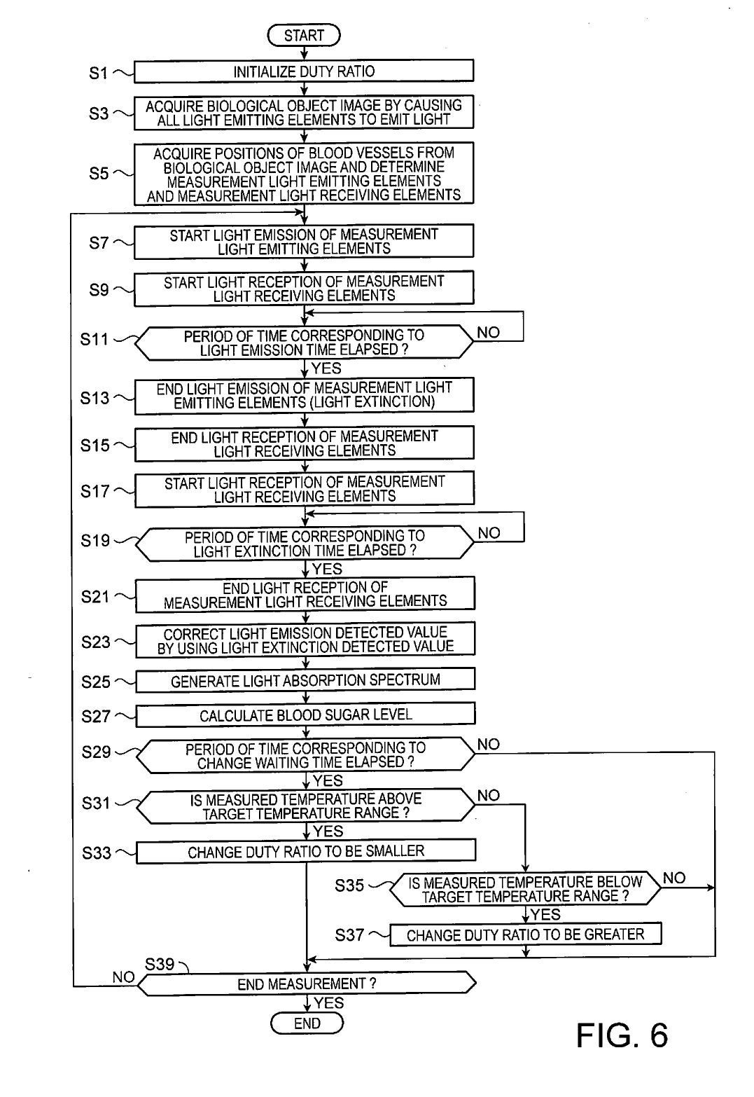

FIG. 6 is a flowchart illustrating a flow of blood sugar level measurement processing. This processing is processing implemented by the processing unit 200 executing the blood sugar level measurement program 302, and starts upon input of a measurement start instruction via the operation unit 102. It is assumed here that the blood sugar level measurement apparatus 10 is appropriately fitted and fixed to the user 2.

First of all, the intermittent irradiation setting unit 208 performs an initialization to set the duty ratio to a predetermined initial value (for example, D=0.5) (step S1). Next, the measurement element selecting unit 202 acquires a biological object image by causing all of the light emitting elements 53 of the light emitting unit 110 to simultaneously emit light (step S3), then acquires positions of blood vessels from the acquired biological object image, and determines measurement light emitting elements and measurement light receiving elements (step S5).

Subsequently, the light emission control unit 204 causes the measurement light emitting elements to start emitting light (step S7), and the light reception control unit 210 causes the measurement light receiving elements to start receiving light (step S9). Then, if a period of time corresponding to the light emission time Ta elapses from the start of light emission of the measurement light emitting elements (YES in step S11), the light emission control unit 204 causes the measurement light emitting elements to finish emitting light (light extinction) (step S13), and the light reception control unit 210 causes the measurement light receiving elements to finish receiving light (step S15). The detected values obtained by light reception are stored as the light emission detected value data 318.

Next, the light reception control unit 210 causes the measurement light receiving elements to start receiving light (step S17). After that, if a period of time corresponding to the light extinction time Tb elapses from the light extinction of the measurement light emitting elements (YES in step S19), the light reception control unit 210 causes the measurement light receiving elements to finish receiving light (step S21). The detected values obtained by light reception are stored as the light extinction detected value data 320. Then, the detected value correction unit 212 performs, with respect to each measurement light receiving element, correction of the light emission detected value by subtracting the light extinction detected value from the light emission detected value (step S23). At this time, if the light emission time Ta and the light extinction time Tb are different, in order to obtain a light extinction detected value corresponding to the light emission time Ta, a value obtained by multiplying the light extinction detected value by Ta/Tb is subtracted from the light emission detected value.

Next, the blood sugar level calculation unit 214 calculates a light absorption spectrum based on the corrected light emission detected value of each measurement light receiving element (step S25), and calculates a blood sugar level from the light absorption spectrums (step S27).

Subsequently, the intermittent irradiation setting unit 208 performs control so as to change the duty ratio. To be specific, if a predetermined change waiting time (for example, 5 minutes) elapses from the previous change of the duty ratio (YES in step S29), the intermittent irradiation setting unit 208 performs comparison between the temperature measured by the temperature measurement unit 206 and a predetermined target temperature range. If the measured temperature is above the target temperature range (YES in step S31), the intermittent irradiation setting unit 208 reduces the duty ratio D so as to extend the light extinction time Tb (step S33). If, on the other hand, the measured temperature is below the target temperature range (NO in step S31 and YES in step S35), the intermittent irradiation setting unit 208 increases the duty ratio D so as to shorten the light extinction time Tb (step S37). If the measured temperature is within the target temperature range (NO in step S35), the intermittent irradiation setting unit 208 does not change the duty ratio D.

After that, a determination is made as to whether to end the blood sugar level measurement by determining whether a measurement end instruction has been input via the operation unit 102. If it is determined that the blood sugar level measurement should not be ended (NO in step S39), the processing returns to step S7. If it is determined that the blood sugar level measurement should be ended (YES in step S39), the processing ends.

Advantageous Effects

As described above, the blood sugar level measurement apparatus 10 according to the present embodiment performs control such that the light emitting elements 53 of the light emitting unit 110 periodically repeat the light emission state and the light extinction state. With this configuration, the temperature of the biological object increases due to irradiation with light during the light emission state, but the temperature of the biological object decreases due to the absence of irradiation with light during the light extinction state. As a result, it is possible to suppress the increase in the blood temperature due to irradiation with light, and improve the accuracy of measuring a component (glucose concentration, or in other words, blood sugar level) in the blood, as compared to the case of continuous irradiation with light.

Variations

Embodiments in which the invention can be applied are not limited to the embodiment described above, and it is of course possible to make changes as appropriate without departing from the scope of the invention.

(A) Measurement of Temperature of Biological Object

The temperature measurement unit 206 is configured to measure the temperature of the biological object by acquiring the temperature measured by the temperature sensor 60, but may be configured to measure the temperature of the biological object in an estimated manner from the detected values of the light receiving elements 59. Due to the temperature dependence of photodiodes serving as the light receiving elements 59, output current values in a light blocking state in which light is not allowed to be incident, or in other words, light extinction detected values vary depending on the temperature. For this reason, a configuration is possible in which a table representing a correspondence between temperature and light extinction detected value is generated and stored in advance, and the temperature measurement unit 206 measures the temperature in an estimated manner from the light extinction detected values by referring to the table.

(B) Change of Duty Ratio

Also, in the embodiment described above, the duty ratio is changed by varying the light extinction time Tb, with the light emission time Ta being fixed, but the light emission time Ta may be varied.

(C) Component to be Measured

In the embodiment described above, glucose concentration in the blood, or in other words, blood sugar level is used as the component to be measured, but it is also possible to measure the component concentration of other sugars such as sucrose and lactose, as well as the component concentration of other substances (protein and lipid). Alternatively, urine may be used as the component to be measured, and uric acid level may be measured by using measurement light with bluish violet light emission wavelengths.

(D) Component Measurement Apparatus

The invention may be applied to an apparatus that optically measures a component of a liquid contained in a container such as a cuvette. With this apparatus as well, the container is placed in a dark room or a dark box, and then irradiated with measurement light, and thus the same problems as the problems of the invention described above occur, but the problems can be solved by the invention. In this case, the test object to be measured may be blood collected from a biological object, or may be any other body fluid such as urine.

The entire disclosure of Japanese Patent Application No. 2014-197034, filed Sep. 26, 2014 is hereby incorporated herein by reference

What is claimed is:

1. A component measurement apparatus comprising:
a light emitting unit that emits light toward a test object;
a light receiving unit that receives light that has been emitted by the light emitting unit and has been reflected within or has passed through the test object;
a light emission control unit that controls the light emitting unit so as to repeat a light emission state and a light extinction state; and
a measurement unit that measures a component in the test object by using a result of light reception by the light receiving unit.

2. The component measurement apparatus according to claim 1,
wherein the light emission control unit performs control such that a ratio of duration of the light emission state to a total duration of the light emission state and the light extinction state is within a range of 0.1 or more and less than 1.0.

3. The component measurement apparatus according to claim 2, further comprising:
a temperature measurement unit that measures a temperature of the test object by using a light extinction detected value, which is a result of light reception by the light receiving unit during the light extinction state,
wherein the light emission control unit controls the ratio according to the measured temperature.

4. The component measurement apparatus according to claim 2,
wherein the light receiving unit includes a light receiving element, and measures the temperature from a light extinction detected value, which is a result of light reception by the light receiving unit during the light extinction state, by referring to a table indicating a relationship between temperature and output current of the light receiving element, and
the light emission control unit controls the ratio according to the measured temperature.

5. The component measurement apparatus according to claim 3,
wherein if the measured temperature is above a predetermined range, the ratio is reduced, and
if the measured temperature is below the predetermined range, the ratio is increased.

6. The component measurement apparatus according to claim 3,
wherein if the measured temperature is within a predetermined range, the ratio is reduced as the measured temperature approaches an upper limit of the range, and is increased as the measured temperature approaches a lower limit of the range.

7. The component measurement apparatus according to claim 1,
wherein the light emission control unit performs control such that duration of a single instance of the light emission state is within a range from 0.01 seconds to 10 minutes.

8. The component measurement apparatus according to claim 1, further comprising:
a correction unit that corrects a light emission detected value, which is a result of light reception by the light receiving unit during the light emission state, by using a light extinction detected value, which is a result of light reception by the light receiving unit during the light extinction state.

9. The component measurement apparatus according to claim 8,
wherein the correction unit corrects the light emission detected value by subtracting the light extinction detected value from the light emission detected value.

10. The component measurement apparatus according to claim 1,
wherein the test object is blood in a biological object,
the light emitting unit emits light including near infrared rays, and
the measurement unit acquires a blood sugar level in the blood.

11. A component measurement method comprising:
controlling a light emitting unit that emits light toward a test object, so as to repeat a light emission state and a light extinction state; and
measuring a component in the test object by using a result of light reception by a light receiving unit that receives light that has been emitted by the light emitting unit and has been reflected within or has passed through the test object.
